(19) United States
(12) Patent Application Publication
(10) Pub. No.: US 2006/0270723 A1
(43) Pub. Date: Nov. 30, 2006

(54) Process for preparing amorphous valsartan

(76) Inventors: Tamas Koltai, Natanya (IL); Omer Malachi, Rehovot (IL); Nisim Sasson, Or Akiva (IL); Amir Gold, Herzelia (IL)

Correspondence Address: KENYON & KENYON LLP, ONE BROADWAY, NEW YORK, NY, 10004, US

(21) Appl. No.: 11/330,519

(22) Filed: Jan. 11, 2006

Related U.S. Application Data
(60) Provisional application No. 60/642,955, filed on Jan. 11, 2005.

(51) Int. Cl. A61K 31/4184

(52) U.S. Cl. 514/381

(57) ABSTRACT

The present invention provides a process for the preparation of amorphous valsartan.

This application claims the benefit of priority to U.S. Provisional Patent Application No. 60/642,955, filed on Jan. 11, 2005.

### BACKGROUND OF THE INVENTION

Valsartan, also known as (S)—N-(1-Carboxy-2-methyl-prop-1-yl)-N-pentanoyl-N-[2′-(1H-tetrazol-5-yl)biphenyl-4-ylmethyl]-amine, has the following structure:

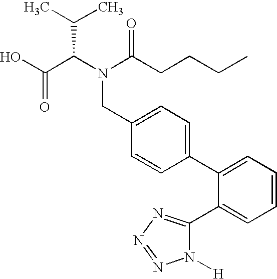

| | |
|---|---|
| Formula | C₂₄H₂₉N₅O₃ |
| Molecular Mass | 435.52 |
| Exact Mass | 435.227040 |
| Composition | C 66.19% H 6.71% N 16.08% O 11.02 |
| Melting Range | 105-110° C. |

and is marketed as the free acid under the name DIOVAN. DIOVAN is prescribed as oral tablets in dosages of 40 mg, 80 mg, 160 mg and 320 mg of valsartan.

Valsartan and/or its intermediates are disclosed in various references, including: U.S. Pat. Nos. 5,399,578, 5,965,592, 5,260,325, 6,271,375, WO 02/006253, WO 01/082858, WO 99/67231, WO 97/30036, Peter Bühlmayer, et al., Bioorgan. & Med. Chem. Let., 4(1) 29-34 (1994), Th. Moenius, et al., J. Labelled Cpd. Radiopharm., 43(13) 1245-1252 (2000), and Qingzhong Jia, et al., Zhongguo Yiyao Gongye Zazhi, 32(9) 385-387 (2001), all of which are incorporated herein by reference.

Valsartan is an orally active specific angiotensin II antagonist acting on the AT1 receptor subtype. Valsartan is prescribed for the treatment of hypertension. U.S. Pat. No. 6,395,728 is directed to use of valsartan for treatment of diabetes related hypertension. U.S. Pat. Nos. 6,465,502 and 6,485,745 are directed to treatment of lung cancer with valsartan. U.S. Pat. No. 6,294,197 is directed to solid oral dosage forms of valsartan. These patents are incorporated herein by reference.

The present invention relates to the solid state physical properties of valsartan. These properties can be influenced by controlling the conditions under which valsartan is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others. A particular polymorphic form may also give rise to distinct spectroscopic properties that may be detectable by powder X-ray crystallography, solid state ¹³C NMR spectrometry and infrared spectrometry.

U.S. Pat. No. 5,399,578, incorporated herein by reference, in Example 16, obtains valsartan and discloses: “melting interval 105-115 (from ethyl acetate).”

In the Merck Index, 12th edition, 1996, p. 1691, n. 10051, valsartan is described as “crystals from diisopropyl ether, mp 116-117° C.” The Merck Index may be reciting the product of example 37 of EP 0 443 983, which is in German. The product is not otherwise characterized by the Merck Index.

In J. of Labelled Compounds and Radiopharmaceuticals, 2000, 43, 1245-1252 on page 1249 (synthesis of [¹⁴C₂] valsartan 2), there is a description of the preparation of valsartan by crystallization from a 1:1 mixture of ethyl acetate and hexane. Repetition of this procedure led to a sample with X-Ray powder diffraction pattern as depicted in FIG. 1 (bottom pattern). The pattern in FIG. 1 shows a diffuse X-Ray diffraction, which indicates presence of an amorphous material.

WO 02/06253 also discloses amorphous form of valsartan: “The X-ray diffraction diagram consists essentially of a very broad, diffuse X-ray diffraction; the free acid is therefore characterized as almost amorphous under X-ray. The melting point linked with the measured melting enthalpy of 12 kJ/mol [approximately 28 J/g] unequivocally confirms the existence of a considerable residual arrangement in the particles or structural domains for the free acid valsartan. There is a need for more stable, e.g. crystalline forms of valsartan.” The WO 02/06253 then goes on to disclose salts of valsartan in crystalline form.

WO 04/083192 discloses purely amorphous form of valsartan.

There is a need in the art for additional processes for preparation of valsartan amorphous form. Amorphous form often has greater bioavailability than crystalline forms and may be more suitable for formulation of an active pharmaceutical ingredient when greater bioavailability is desired.

### SUMMARY OF THE INVENTION

The present invention provides a process for preparing amorphous valsartan having a DSC thermogram that lacks endothermic peaks above about 1 J/g in the region of from about 80° C. to about 100° C. comprising: preparing a solution of valsartan in a solvent selected from the group consisting of: C₁₋₃ alcohols, C₂₋₄ esters, C₃₋₅ ethers, ketones, C₁₋₅ amides, DMSO, acetonitrile, toluene and a mixture thereof with water, and removing the solvent.

### BRIEF DESCRIPTION OF THE FIGURES

FIG. 1 is three different X-Ray powder diffraction (“XRPD”) patterns of valsartan.

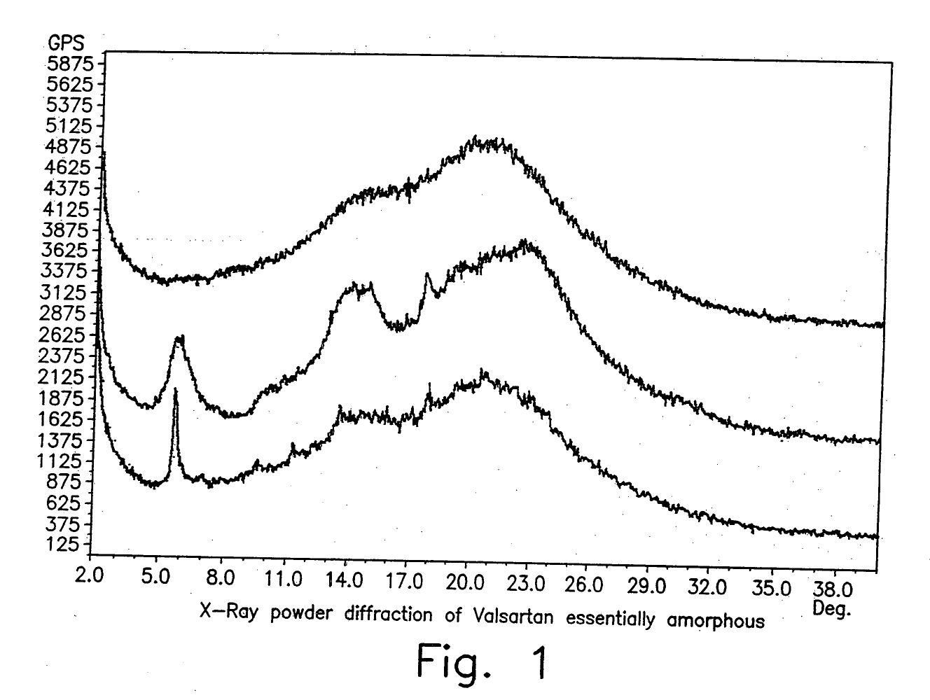

FIG. 2 is an X-Ray powder diffraction of valsartan purely amorphous.

FIG. 3 is a DSC thermogram of valsartan purely amorphous.

FIG. 4 is an X-Ray powder diffraction (“XRPD”) pattern of amorphous valsartan prepared by the process of the present invention.

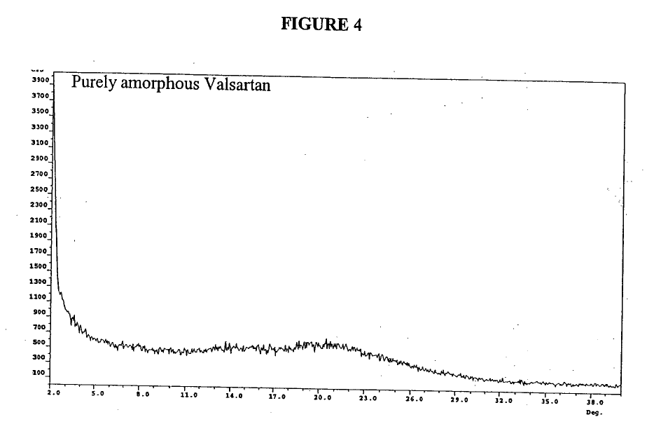

### DETAILED DESCRIPTION OF THE INVENTION

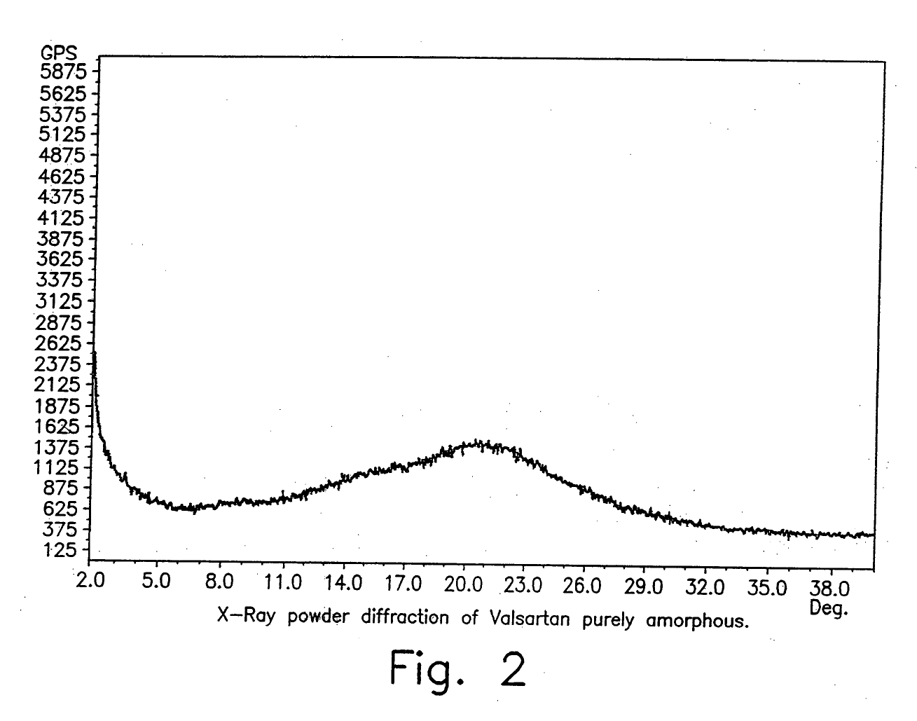

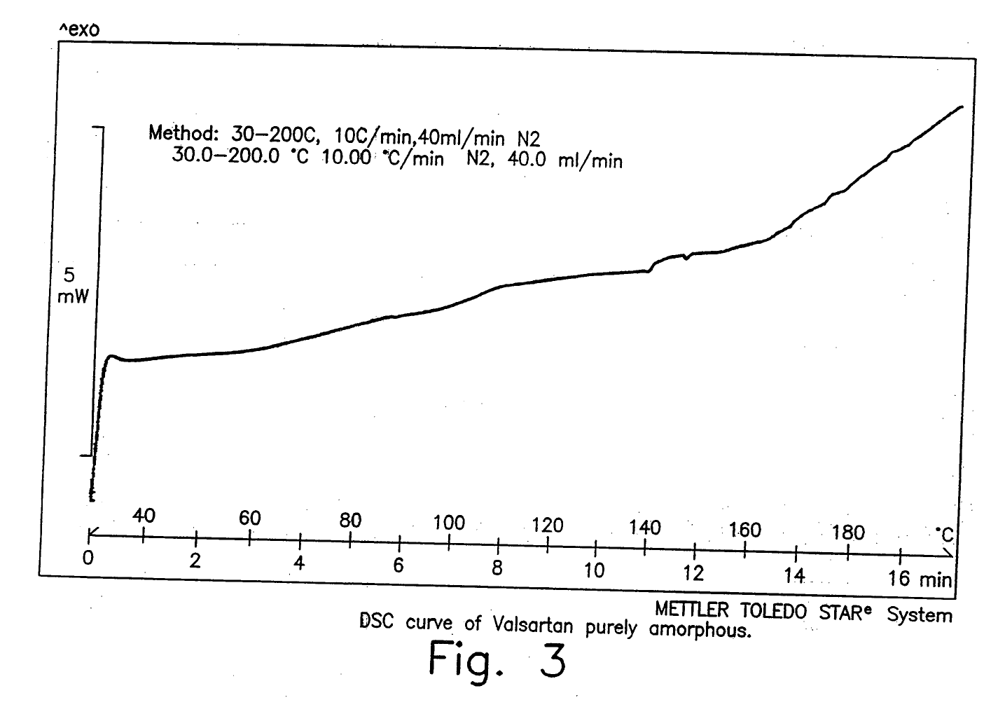

An amorphous Valsartan that is substantially free of crystalline forms is herein referred to as “valsartan purely amorphous.” FIG. 2 illustrates an XRPD pattern for this form, where the halo shape of the pattern illustrates the substantial absence of crystalline structure. Peaks and bumps are particularly missing in the regions characteristic of crystalline form. Additionally, the “valsartan purely amorphous” has a DSC thermogram as substantially depicted in FIG. 3. The DSC thermogram lacks endothermic peaks, such as those above about 1 J/g, preferably those above about 0.5 J/g, in the region of from about 80° C. to about 100° C.

The present invention provides a process for the preparation of amorphous valsartan having a DSC thermogram that lacks endothermic peaks above about 1 J/g in the region of from about 80° C. to about 100° C. This process comprises dissolving valsartan in a solvent selected from the group consisting of: C₁₋₃ alcohols, C₂₋₄ esters, C₃₋₅ ethers, ketones, C₁₋₅ amides, DMSO, acetonitrile, toluene and a mixture thereof with water, followed by solvent removal.

Preferably, the solvent is selected from a group consisting of: DMF, acetonitrile, DMSO, methanol, ethanol, IPA, ethyl acetate, n-butyl acetate, acetone, methyl ethyl ketone, THF, isopropyl ether, dioxane, t-butyl methyl ether and toluene.

Most preferably the solvent is ethyl acetate.

The solution of valsartan in a solvent may be prepared by various methods. For example, when combining valsartan in a solvent to form a solution, warming of the mixture may be carried out to completely dissolve the starting material. If warming does not clarify the mixture, the mixture may be diluted or filtered. To filter, the hot mixture may be passed through paper, glass fiber or other membrane material, or a clarifying agent such as celite. Depending upon the equipment used and the concentration and temperature of the solution, the filtration apparatus may need to be preheated to avoid premature crystallization.

The amount of solvent used is dependent on the introduced quantity of valsartan. The solvent is added until valsartan is fully dissolved. Whenever ethyl acetate is used as a solvent, valsartan has a concentration of from about 0.05 kg/L to about 1 kg/L.

Removing the solvent can be performed using vacuum drying or spray drying.

Vacuum drying broadly refers to processes involving removal of liquid material from a solution or mixture under air pressure below atmospheric pressure. The process of the present invention may preferably employ vacuum drying at a pressure of less than about 100 mm Hg, more preferably less than about 40 mm Hg.

Alternatively, the solution may be spray dried. Spray drying broadly refers to processes involving breaking up liquid mixtures into small droplets (atomization) and rapidly removing solvent from the mixture. In a typical spray drying apparatus, there is a strong driving force for evaporation of solvent from the droplets, which may be provided by providing a drying gas. Spray drying processes and equipment are described in Perry's Chemical Engineer's Handbook, pp. 20-54 to 20-57 (6th ed. 1984) and Remington: The Science and Practice of Pharmacy, 19th ed., vol. 11, pg. 1627, which are herein incorporated by reference.

By way of non-limiting example only, the typical spray drying apparatus comprises a drying chamber, atomizing means for atomizing a solvent-containing feed into the drying chamber, a source of drying gas that flows into the drying chamber to remove solvent from the atomized-solvent-containing feed, an outlet for the products of drying, and product collection means located downstream of the drying chamber. Examples of such apparatuses include Niro Models PSD-1, PSD-2 and PSD-4 (Niro A/S, Soeborg, Denmark). Typically, the product collection means includes a cyclone connected to the drying apparatus. In the cyclone, the particles produced during spray drying are separated from the drying gas and evaporated solvent, allowing the particles to be collected. A filter may also be used to separate and collect the particles produced by spray drying.

The processes of the present invention may preferably employ spray drying with an inlet temperature of from about 40° C. to about 140° C. Most preferably the inlet temperature is at least about 120° C.

The spray drying may preferably be conducted with a solution feed rate of less than about 5 L/h, more preferably about 1 L/h.

The spray drying may preferably be conducted with an outlet temperature of below the inlet temperature, more preferably below about 90° C., and most preferably about 65° C. The drying gas used in the process of the present invention may be any suitable gas, although inert gases such as nitrogen, nitrogen-enriched air, and argon are preferred. The valsartan product produced by spray drying may be recovered by techniques commonly used in the art, such as using a cyclone or a filter.

The product of the present invention is amorphous valsartan having a DSC thermogram that lacks endothermic peaks above about 1 J/g in the region of from about 80° C. to about 100° C.

The starting material used for the processes of the present invention may be any crystalline or amorphous form of valsartan, including any solvates and hydrates. With processes where valsartan goes into solution, the form of the starting material is of minimal relevance since any solid state structure is lost in solution.

Pharmaceutical compositions of the present invention contain valsartan purely amorphous, optionally in mixture with other form(s) of valsartan. The valsartan prepared by the processes of the present invention are ideal for pharmaceutical formulation. In addition to the active ingredient(s), the pharmaceutical compositions of the present invention may contain one or more excipients. Excipients are added to the composition for a variety of purposes.

The pharmaceutical compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages can be conveniently presented in unit dosage form and prepared by any of the methods well-known in the pharmaceutical arts.

The active ingredient and excipients may be formulated into compositions and dosage forms according to methods known in the art. The solid oral dosage forms disclosed in U.S. Pat. Nos. 6,485,745 and 6,395,728 may be used as a guidance. The dosages and formulation of DIOVAN may also be used for guidance. The dosage is preferably from about 10 mg to about 1280 mg, more preferably from about 20 mg to about 640 mg, and most preferably from about 40 mg to about 320 mg.

### Instruments

X-ray powder diffraction data were obtained using by method known in the art using a SCINTAG powder X-Ray diffractometer model X'TRA equipped with a solid state detector. Copper radiation of 1.5418 Å was used. A round aluminum sample holder with round zero background quartz plate, with cavity of 25(diameter)*0.5(dept) mm.

DSC analysis was done using a Mettler 821 Stare. The weight of the samples is about 5 mg., the samples were scanned at a rate of 10° C./min from 30° C. to 200° C. The oven was constantly purged with nitrogen gas at a flow rate of 40 ml/min. Standard 40 ml aluminum crucibles covered by lids with three holes were used.

### Procedures

### EXAMPLE 1

A solution of 10% valsartan in ethyl acetate was prepared (Kg/L). The solution was spray dried on a “BUCHI” Mini spray dryer B-290 using a spry nozzle. The inlet temperature was set to 120° C. The solution feed rate was about 1 L/h and the draying gas flow was 27 m³/h. The outlet temperature was maintained at 65° C. The product was collected at the bottom of the cyclone and sent to XRD analysis. The sample was found to be amorphous valsartan having a DSC thermogram that lacks endothermic peaks above about 1 J/g in the region of from about 80° C. to about 100° C.

### EXAMPLE 2

A solution of 20% valsartan in ethyl acetate was prepared (Kg/L). The solution was spray dried on a “BUCHI” Mini spray dryer B-290 using a spry nozzle. The inlet temperature was set to 120° C. The solution feed rate was about 1 L/h and the draying gas flow was 27 m³/h. The outlet temperature was maintained at 60° C. The product was collected at the bottom of the chamber and sent to XRD analysis. The sample was found to be amorphous valsartan having a DSC thermogram that lacks endothermic peaks above about 1 J/g in the region of from about 80° C. to about 100° C.

### EXAMPLE 3

A solution of 40% valsartan in ethyl acetate with 5% of water was prepared (Kg/L). The solution was spray dried on a “BUCHI” Mini spray dryer B-290 using a spry nozzle. The inlet temperature was set to 90° C. The solution feed rate was about 0.15 L/h and the draying gas flow was 38 m³/h. The outlet temperature was maintained at 61° C. The product was collected at the bottom of the chamber and sent to XRD analysis. The sample was found to be amorphous valsartan having a DSC thermogram that lacks endothermic peaks above about 1 J/g in the region of from about 80° C. to about 100° C.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications.

We claim:

1. A process for preparing amorphous valsartan having a DSC thermogram that lacks endothermic peaks above about 1 J/g in the region of about 80° C. to about 100° C. comprising:
a) preparing a solution of valsartan in a solvent selected from the group consisting of C₁₋₃ alcohols, C₂₋₄ esters, C₃₋₅ ethers, ketones, C₁₋₅ amides, DMSO, acetonitrile, toluene and a mixture thereof with water; and
b) removing the solvent.

2. The process of claim 1, wherein the solvent is selected from a group consisting of: DMF, acetonitrile, DMSO, methanol, ethanol, IPA, ethyl acetate, n-butyl acetate, acetone, methyl ethyl ketone, THF, isopropyl ether, dioxane, t-butyl methyl ether and toluene.

3. The process of claim 1, wherein the solution has a valsartan concentration of from about 0.05 kg/L to about 1 kg/L.

4. The process of claim 1, wherein the step a) comprises warming the solvent to promote complete dissolution of the valsartan.

5. The process of claim 1, wherein the solvent is removed by vacuum drying.

6. The process of claim 5, wherein the vacuum drying is conducted at a pressure of less than about 100 mm Hg.

7. The process of claim 6, wherein the pressure is less than about 40 mm Hg.

8. The process of claim 1, wherein the solvent is removed by spray drying.

9. The process of claim 8, wherein the spray drying is conducted with an inlet temperature of from about 40° C. to about 140° C.

10. The process of claim 9, wherein the inlet temperature is at least about 120° C.

11. The process of claim 8, wherein the spray drying is conducted with a solution feed rate of less than about 5 L/h.

12. The process of claim 11, wherein the solution feed rate is about 1 L/h.

13. The process of claim 8, wherein the spray drying is conducted with an outlet temperature below that of an inlet temperature.

14. The process of claim 13, wherein the outlet temperature is below about 90° C.

15. The process of claim 15, wherein the outlet temperature is about 65° C.
